(19) United States
(12) Patent Application Publication
(10) Pub. No.: US 2012/0142986 A1
(43) Pub. Date: Jun. 7, 2012

(54) PROCESS FOR PRODUCING AROMATIC HYDROCARBON AND TRANSITION-METAL-CONTAINING CRYSTALLINE METALLOSILICATE CATALYST FOR USE IN THE PRODUCTION PROCESS

(75) Inventors: Akihiro Okabe, Narashino-shi (JP); Yoshimichi Namai, Funabashi-shi (JP); Hideyuki Ito, Singapore (SG); Satoshi Akiyama, Yokohama-shi (JP); Michiaki Umeno, Chiba-shi (JP); Takashi Ono, Ichihara-shi (JP); Toru Nishimura, Chiba-shi (JP)

(73) Assignees: AGENCY FOR SCIENCE TECHNOLOGY AND RESEARCH, Singapore (SG); MITSUI CHEMICALS, INC., Tokyo (JP)

(21) Appl. No.: 13/389,571

(22) PCT Filed: Aug. 4, 2010
(86) PCT No.: PCT/JP2010/063161

§ 371 Date: Feb. 8, 2012

(30) Foreign Application Priority Data
Aug. 12, 2009 (JP) 2009-187454

(51) Int. Cl. C07C 2/46; B01J 21/06

(52) U.S. Cl. 585/418; 502/240

(57) ABSTRACT

Provided is a process for producing an aromatic hydrocarbon efficiently at high yield from a lower hydrocarbon containing methane as a major component, and such a process for producing an aromatic hydrocarbon includes the step of reacting a lower hydrocarbon containing methane as a major component in the presence of a transition-metal-containing crystalline metallosilicate catalyst which is obtainable by supporting 5 to 25 parts by weight of a transition metal (X) on 100 parts by weight of a modified crystalline metallosilicate obtainable by subjecting a crystalline metallosilicate to a series of treatment (A) including a step (i) of eliminating part of a metal from the crystalline metallosilicate and a silylation step (ii).

### TECHNICAL FIELD

The present invention relates to a process for producing an aromatic hydrocarbon from a lower hydrocarbon containing methane as a major component. More particularly, the present invention relates to a process for efficiently producing an aromatic hydrocarbon useful as a chemical industrial raw material from a lower hydrocarbon containing methane as a major component in the presence of a transition-metal-containing crystalline metallosilicate catalyst. Furthermore, the present invention relates to a transition-metal-containing crystalline metallosilicate catalyst used in the above process.

### BACKGROUND ART

Conventionally, aromatic hydrocarbons represented by benzene, toluene, xylene, etc. have been produced for the most part as a by-product of gasoline production in petroleum refinery industry or as a by-product of ethylene production in petrochemical industry. Because the aromatic hydrocarbons are not targeted products in both cases, the yields of the aromatic hydrocarbons based on a starting raw material, crude oil, are not high. As a production process in which the aromatic hydrocarbons are targeted products, processes to use a light component derived from crude oil as a raw material were developed, a part of which have been commercialized, but the production amount is still small.

Meanwhile, it is said that natural gas reserves in the whole world are about 6000 TCF, but most amount thereof has not been effectively used. A technique of producing the aromatic hydrocarbons from methane, which is a major component of the natural gas, can give high added value to the abundant natural gas, and can convert the raw material source of the aromatic hydrocarbons, which are important materials in chemical industry, into a non-crude-oil resource. The commercialization of this process is thus desired.

An example which has been studied most intensively of a catalyst widely known to have excellent properties for directly producing the aromatic hydrocarbons from methane as a raw material is a catalyst comprising a molybdenum-supported zeolite catalyst discovered in 1993 by L. Wang, et al. (Non-Patent Document 1). As a catalyst capable of directly producing the aromatic hydrocarbons from methane with good efficiency disclosed in conventional techniques, a crystalline metallosilicate supporting a transition metal, in particular, a MFI type zeolite or a MWW type zeolite supporting molybdenum, tungsten or rhenium has been widely known.

In the reaction to generate the aromatic hydrocarbons from methane, in general, the reaction equilibrium is favorable at a high temperature side for the generation of the aromatic hydrocarbons. For example, in the reaction to generate benzene from methane, the equilibrium conversion is thermodynamically estimated to be about 5% at 600° C., about 11% at 700° C., and about 20% at 800° C. As such, from the limitation imposed by the reaction equilibrium, the efficient production of the aromatic hydrocarbons to a satisfactory extent requires the reaction temperature in this reaction system to be high, e.g., 600° C. or higher, preferably 700 to 750° C. or higher.

The crystalline metallosilicate is an excellent catalyst to produce the aromatic hydrocarbons from the lower hydrocarbon containing methane as a major component. On the other hand, this catalyst has a problem that under high temperature conditions as described above, the catalyst, with partial degradation of the crystal structure, etc., loses its performance. Thus, an industrial problem to be solved is enabling the crystalline metallosilicate to have improved durability under high temperature reaction conditions, i.e., thermal stability, and lengthened catalyst life. However, no technique has been disclosed concerning the reaction system to produce the aromatic hydrocarbons from the lower hydrocarbon containing methane as a major component to improve the thermal stability of the crystalline metallosilicate thereby effectively lengthening the catalyst life.

Meanwhile, as a method to enable the crystalline metallosilicate to have improved durability under high temperature conditions, it is known to be effective to prevent elimination of a metal from the crystalline metallosilicate.

Specifically, it has been known to employ (1) partial removal of a metal component, (2) surface coating through silylation, phosphorus modification or the like, and (3) ion exchange supporting of an alkali metal, an alkaline earth metal, a rare earth metal or the like.

In the method (1), a metal component which is readily eliminated is removed beforehand, thereby causing a stable metal component which is hardly eliminated to remain. An example in this method is an ultrastable Y zeolite (USY, Patent Document 1). In the method (2), an attack by water molecule to a metal component in the crystalline metallosilicate framework is physically decreased. An example in this method is a phosphorus-modified ZSM-5 zeolite (Non-Patent Document 2). In the method (3), the interaction between a cation of an electropositive element and a metal component is utilized to control an electron density of a [MO₄] unit (M is a metal) within a metallosilicate framework. An example in this method is a rare-earth-substituted Y zeolite for fluid catalytic cracking (REY, Patent Document 2).

The selective metal elimination treatment carried out for the purpose of the method (1) is known to be performable by a method such as a treatment with high-temperature steam and a treatment with a mineral acid such as hydrochloric acid and nitric acid, under appropriate conditions. These treatments cause the hydrolysis of a M-O—Si bond (M is a metal) in the crystalline metallosilicate framework, whereby the metal component is removed, while a site from which the metal has been removed becomes a hydroxyl group (a silanol group), which is left as a silanol nest (a hydroxyl nest). The silanol nest is a lattice defect, and the presence of a remaining silanol group facilitates the hydrolysis.

In response thereto, there has been disclosed that repairing this lattice defect can prevent the metal elimination more effectively (Patent Document 3). In other words, the method (1), when combined with the method (2), can work effectively.

Hexafluorosilicates and silicon tetrachloride, which are silicon based metal-elimination agents, serve also as a silylation agent (for example, Patent Documents 4 and 5, and Non-Patent Document 3). For this reason, they can silylate the silanol nest as soon as the silanol nest arises on the surface of the crystalline metallosilicate as a result of the metal elimination treatment. This method therefore can effectively repair every possible surface defect. In addition, this method, in which a single material can perform the two different treatments by one-pot, is simple, having fewer numbers of steps. In this regard, the silicon based metal-elimination agent can be cited as an ideal method combining the methods (1) and (2). Still, the treatment with the silicon based metal-elimination agent is known to have limitation on the degree of the metal elimination (for example, Non-Patent Document 4), thus failing to ensure the attainment of sufficient effect.

In addition thereto, as a catalyst prepared through the combination of the methods (1) and (3), a rare earth exchange ultrastable Y zeolite (RE-USY, Patent Document 6) has been disclosed. Further, there have been disclosed reaction processes using a catalyst combining the methods (2) and (3), such as La/P/ZSM-5 (Patent Document 7) and Mg/P/ZSM-5 (Patent Document 8).

However, none of the aforementioned techniques to prevent the elimination of a metal from the crystalline metallosilicate under high temperature conditions have been found to be relevant to the improvement of the catalyst life in the reaction to produce the aromatic hydrocarbons from the lower hydrocarbon containing methane as a major component.

On the other hand, Non-Patent Document 5, regarding the reaction to produce benzene from methane, studies the activity decrease due to coking of a molybdenum-supported zeolite catalyst, and discloses examples of applying various treatment methods to a catalyst in the reaction to produce benzene from methane. As a method to reduce coking, this document discloses controlling the acidity of zeolite, and studies various dealumination treatments with respect to zeolite, an example of which is a treatment with ammonium hexafluorosilicate. In the case of the treatment using ammonium hexafluorosilicate, however, the selectivity of benzene is reduced (see FIG. 5). As is clear therefrom, the Non-Patent Document 5 does not suggest the treatment with the silicon based metal-elimination agent is suitable for a catalyst in the reaction to produce the aromatic hydrocarbons from the lower hydrocarbon containing methane as a major component. Furthermore, while the Non-Patent Document 5 discloses reduction of coking, it does not disclose improvement of the thermal stability of the zeolite catalyst itself and lengthening of the catalyst life.

Meanwhile, since methane has extremely lower reactivity compared with other hydrocarbons employed in the above conventional art, the production of the aromatic hydrocarbons from the lower hydrocarbon containing methane as a major component usually employs ZSM-5 having a smaller ratio of silica to alumina (having a larger Al content and a larger amount of acid).

However, it was readily understood that the acid amount and the acid strength are reduced in the methods to modify the crystalline metallosilicate as disclosed in the aforementioned conventional art, and therefore the use of these methods for the reaction of methane was not usually considered. In fact, in the Non-Patent Document 5, the method to modify the crystalline metallosilicate using ammonium hexafluorosilicate involved the reduction of the acid amount and the acid strength (Table 1), consequently lowering the selectivity of benzene (FIG. 5).

In other words, it was difficult even for a skilled person in the art to conceive of applying the aforementioned techniques to the catalyst in the reaction to produce the aromatic hydrocarbons from the lower hydrocarbon containing methane as a major component and achieving a further improvement.

With respect to the reaction to produce the aromatic hydrocarbons from the lower hydrocarbon such as methane, Patent Document 9 discloses the use of a catalyst in which a molybdenum compound or the like is supported on a metallosilicate carrier obtained through silylation modification not accompanying metal elimination, or on a metallosilicate carrier modified with an oxide of an alkali metal or alkali earth metal. Patent Document 10 discloses a technique of selectively passivating the acidity of an outer surface of a ZSM-5 catalyst with an amorphous silica layer. These techniques, however, are not relevant to the thermal stability of the metallosilicate, and even using these techniques fails to achieve sufficient thermal stability of the metallosilicate and realize a longer catalyst life.

### CITATION LIST

### Patent Document

Patent Document 1: U.S. Pat. No. 3,449,070
Patent Document 2: U.S. Pat. No. 4,415,438
Patent Document 3: JP-A-1997-173853
Patent Document 4: U.S. Pat. No. 4,503,023
Patent Document 5: U.S. Pat. No. 5,157,191
Patent Document 6: U.S. Pat. No. 4,938,863
Patent Document 7: JP-A-1999-180902
Patent Document 8: WO-A-2007-043741
Patent Document 9: JP-A-2006-249065
Patent Document 10: JP-A-2004-521070

### Non-Patent Document

Non-Patent Document 1: Catalysis Letters, 1993, Vol. 21, page 35
Non-Patent Document 2: Journal of Catalysis, 2006, Vol. 237, page 267
Non-Patent Document 3: Catalysis Communications, 2008, Vol. 9, page 907
Non-Patent Document 4: Journal of the Chemical Society, Chemical Communications, 1989, page 1908
Non-Patent Document 5: Studies in Surface Science and Catalysis, 2008, 174B, page 1075

### SUMMARY OF THE INVENTION

### Problem to be Solved by the Invention

It is an object of the present invention to provide a process for producing an aromatic hydrocarbon efficiently at high yield from a lower hydrocarbon containing methane as a major component.

### Means for Solving the Problem

The present inventors diligently studied to solve the above problem, and found that the effective combination of a single or plural mollification methods for preventing the elimination of a metal from a crystalline metallosilicate catalyst, and setting a supporting amount of a transition metal and a treatment for supporting an alkali metal and the like solves the problem with the thermal stability of the crystalline metallosilicate catalyst, which is a potential problem arising in the reaction to produce an aromatic hydrocarbon from a lower hydrocarbon containing methane as a major component, and furthermore improves the catalyst life in this reaction. The present invention has been made based on this finding.

That is, the present invention includes the following matters.

[1] A process for producing an aromatic hydrocarbon comprising the step of reacting a lower hydrocarbon containing methane as a major component in the presence of a transition-metal-containing crystalline metallosilicate catalyst which is obtainable by supporting 5 to 25 parts by weight of a transition metal (X) on 100 parts by weight of a modified crystalline metallosilicate obtainable by subjecting a crystalline metallosilicate to a series of treatment (A) comprising a step (i) of eliminating part of a metal from the crystalline metallosilicate and a silylation step (ii).

[2] The process for producing an aromatic hydrocarbon as described in [1], wherein the series of treatment (A) is contacting the crystalline metallosilicate to an aqueous solution of a hexafluorosilicate.

[3] The process for producing an aromatic hydrocarbon as described in [2], wherein the hexafluorosilicate is ammonium hexafluorosilicate.

[4] A process for producing an aromatic hydrocarbon comprising the step of reacting a lower hydrocarbon containing methane as a major component in the presence of a transition-metal-containing crystalline metallosilicate catalyst which is obtainable by supporting a transition metal (X) on a modified crystalline metallosilicate obtainable by subjecting a crystalline metallosilicate to a series of treatment (A) comprising a step (i) of eliminating part of a metal from the crystalline metallosilicate and a silylation step (ii) and a treatment (B) of supporting one kind or two or more kinds of metals (Y) selected from the group consisting of alkali metals, alkaline earth metals and rare earth metals.

[5] The process for producing an aromatic hydrocarbon as described in [4], wherein the series of treatment (A) is contacting the crystalline metallosilicate to an aqueous solution of a hexafluorosilicate.

[6] The process for producing an aromatic hydrocarbon as described in [5], wherein the hexafluorosilicate is ammonium hexafluorosilicate.

[7] The process for producing an aromatic hydrocarbon as described in any one of [4] to [6], wherein the treatment (B) is performed through ion exchange method.

[8] The process for producing an aromatic hydrocarbon as described in any one of [4] to [7], wherein the metal (Y) is an alkaline earth metal.

[9] The process for producing an aromatic hydrocarbon as described in any one of [4] to [8], wherein the metal (Y) is barium.

[10] The process for producing an aromatic hydrocarbon as described in any one of [1] to [9], wherein the crystalline metallosilicate is a MFI-type zeolite or a MWW-type zeolite.

[11] The process for producing an aromatic hydrocarbon as described in any one of [1] to [10], wherein the transition metal (X) is one kind or two or more kinds selected from the group consisting of molybdenum, tungsten and rhenium.

[12] The process for producing an aromatic hydrocarbon as described in any one of [1] to [11], wherein the transition metal (X) is molybdenum.

[13] A transition-metal-containing crystalline metallosilicate catalyst for use in the process for producing an aromatic hydrocarbon as described in [1], which catalyst is obtainable by supporting 5 to 25 parts by weight of a transition metal (X) on 100 parts by weight of a modified crystalline metallosilicate obtainable by subjecting a crystalline metallosilicate to a series of treatment (A) comprising a step (i) of eliminating part of a metal from the crystalline metallosilicate and a silylation step (ii).

[14] A transition-metal-containing crystalline metallosilicate catalyst for use in the process for producing an aromatic hydrocarbon as described in [4], which catalyst is obtainable by supporting a transition metal (X) on a modified crystalline metallosilicate obtainable by subjecting a crystalline metallosilicate to a series of treatment (A) comprising a step (i) of eliminating part of a metal from the crystalline metallosilicate and a silylation step (ii) and a treatment (B) of supporting one kind or two or more kinds of metals (Y) selected from the group consisting of alkali metals, alkaline earth metals and rare earth metals.

### Effect of the Invention

By effectively combining the step of eliminating part of a metal from the crystalline metallosilicate and the silylation step to prevent the elimination of the metal, with setting the supporting amount of a transition metal, and the treatment for supporting an alkali metal and the like, a transition-metal-containing crystalline metallosilicate catalyst having a high durability under high temperature conditions (namely, thermal stability) and a long catalyst life is obtained economically through a simplified operation.

By using the transition-metal-containing crystalline metallosilicate catalyst as a catalyst in the reaction to produce an aromatic hydrocarbon from a lower hydrocarbon containing methane as a major component, the aromatic hydrocarbon can be obtained while maintaining high yield for long hours, and therefore the above process is industrially useful.

### EMBODIMENTS FOR CARRYING OUT THE INVENTION

Hereinafter, a process for producing an aromatic hydrocarbon of the present invention, and a transition-metal-containing crystalline metallosilicate used in the production process are described in detail. Embodiments set forth herein are provided as detailed description for better understanding of the gist of the present invention, and thus should not be construed as limiting the present invention unless otherwise noted.

### [Process for Producing Aromatic Hydrocarbon]

The first process for producing an aromatic hydrocarbon of the present invention comprises reacting a lower hydrocarbon containing methane as a major component in the presence of the first transition-metal-containing crystalline metallosilicate catalyst (hereinafter, also called a “metallosilicate catalyst (1)”).

The second process for producing an aromatic hydrocarbon of the present invention comprises reacting a lower hydrocarbon containing methane as a major component in the presence of the second transition-metal-containing crystalline metallosilicate catalyst (hereinafter, also called a “metallosilicate catalyst (2)”).

Hereinafter, the metallosilicate catalysts (1) and (2) are described. When the metallosilicate catalyst (1) and the metallosilicate catalyst (2) are not particularly distinguished from each other, they are also described as a “transition-metal-containing crystalline metallosilicate catalyst”.

### [Metallosilicate Catalysts (1) and (2)]

The metallosilicate catalyst (1) is obtainable by supporting 5 to 25 parts by weight of a transition metal (X) on 100 parts by weight of a modified crystalline metallosilicate obtainable by subjecting a crystalline metallosilicate to a series of treatment (A) comprising a step (i) of eliminating part of a metal from the crystalline metallosilicate and a silylation step (ii). As the modified crystalline metallosilicate, it is preferable to use a modified crystalline metallosilicate obtainable by subjecting the crystalline metallosilicate to the series of treatment (A), and further a treatment (B) of supporting one kind or two or more kinds of metals (Y) selected from the group consisting of alkali metals, alkaline earth metals and rare earth metals.

The metallosilicate catalyst (2) is obtainable by supporting a transition metal (X) on a modified crystalline metallosilicate obtainable by subjecting a crystalline metallosilicate to a series of treatment (A) comprising a step (i) of eliminating part of a metal from the crystalline metallosilicate and a silylation step (ii) and a treatment (B) of supporting one kind or two or more kinds of metals (Y) selected from the group consisting of alkali metals, alkaline earth metals and rare earth metals.

Hereinafter, the treatment (A), the treatment (B), and the treatment for supporting the transition metal (X) are described.

### <Treatment (A)>

The treatment (A) is a series of treatment comprising a step (i) of eliminating part of a metal from a crystalline metallosilicate and a silylation step (ii).

Since the presence of another treatment between the step (i) and the step (ii) is considered to possibly inhibit the silylation and the elimination of part of the metal, the series of the treatment (A) refers to treatments that involve the two steps without another treatment therebetween.

Examples of the crystalline metallosilicate include zeolite, aluminosilicate, gallosilicate, galloaluminosilicate, borosilicate and phophoaluminosilicate; preferred examples are zeolite and aluminosilicate; and more preferred examples are a MFI type zeolite represented by a ZSM-5 type zeolite, and a MWW type zeolite represented by a MCM-22 type zeolite. These may be used in a single kind or in a mixture of two or more kinds. The crystalline metallosilicate may be a commercially available product, or may be synthesized from inorganic compound raw materials by known methods.

When using the above zeolites, the ratio of silica to alumina before carrying out the treatment (A) is preferably smaller in a range that does not impair the stability of the zeolite structure. The ratio is usually not more than 100, preferably not more than 55, more preferably not more than 45, still more preferably not more than 35, particularly preferably not more than 30. A lower limit of the ratio of silica to alumina is not particularly limited, but is usually about 25.

The process for producing an aromatic hydrocarbon of the present invention uses a lower hydrocarbon containing methane as a major component. Since the reactivity of methane is extremely lower than that of other lower hydrocarbons, it is preferable to use a crystalline metallosilicate having a smaller ratio of silica to alumina, as described above.

A counterion of the crystalline metallosilicate is not particularly limited, but is preferably an ammonium type and a proton type, more preferably an ammonium type.

As a method for eliminating part of the metal from the crystalline metallosilicate, commonly known methods can be mentioned without particular limitation, for example, a treatment with high-temperature steam; a treatment using mineral acids such as hydrochloric acid, nitric acid and sulfuric acid; a treatment using ethylene diamine tetraacetic acid; a treatment using hexafluorosilicates; and a treatment using silicon tetrachloride.

As a method for silylation, commonly known methods are used without particular limitation: for example, there can be mentioned treatments using alkoxysilanes such as tetraethoxy silane and aminopropyl triethoxy silane; hydrosilanes such as triethoxy silane and trimethoxy silane; silazanes such as hexamethyl disilazane and nonamethyl trisilazane; and silyl halide compounds such as ammonium hexafluorosilicate, silicon tetrachloride and chlorotrimethyl silane.

Exemplary procedures for the series of treatment (A) comprising the step (i) and the step (ii) include (1) a procedure in which the metal elimination and the silylation are divided into two stages, and the metal elimination is carried out at first and then the silylation is carried out; (2) a procedure in which the metal elimination and the silylation are divided into two stages, and the silylation is carried out at first and then the metal elimination is carried out; and (3) a procedure in which the metal elimination and the silylation are simultaneously carried out by one-pot. Any of these procedures may be employed, but the procedures (1) and (3) are preferable, in which silylation treatment can be carried out with respect to the silanol nest that is generated as a result of the metal elimination; and the procedure (3) is particularly preferable, which has a fewer number of steps and in which the silylation treatment can be carried out on the surface of the zeolite having the silanol nest that is generated as a result of the metal elimination.

As a method suitable for the procedure (3), a treatment using a silicon based metal-elimination agent can be mentioned. Preferred examples of the treatment are a treatment using fluorosilyl compounds and a treatment using chlorosilyl compounds; more preferred examples of the treatment are a treatment using hexafluorosilicates and a treatment using silicon tetrachloride; a still more preferred example of the treatment is a treatment using hexafluorosilicates; and a particularly preferred example of the treatment is a treatment using ammonium hexafluorosilicate.

As a method for carrying out the treatment using the silicon based metal-elimination agent, exemplary methods are a method of contacting the crystalline metallosilicate with a solution of the agent, a method of exposing the crystalline metallosilicate to vapor of the agent, and a method of mixing the crystalline metallosilicate with the agent in a solid state followed by the calcining of the mixture (solid state substitution method). The method of contacting with a solution usually employs a solvent compatible with the silicon based metal-elimination agent. When the silicon based metal-elimination agent is a hexafluorosilicate, exemplary employable methods are a method of contacting with an aqueous solution, and the solid state substitution method. When the silicon based metal-elimination agent is silicon tetrachloride, exemplary employable methods are a method of contacting with a solution, and a method of exposing to vapor.

As the treatment (A), a preferable treatment is contacting the crystalline metallosilicate with an aqueous solution of a hexafluorosilicate, in which case the use of ammonium hexafluorosilicate as a hexafluorosilicate is more preferable.

### <Treatment (B)>

The treatment (B) is a treatment of supporting one kind or two or more kinds of metals (Y) selected from the group consisting of alkali metals, alkaline earth metals and rare earth metals which treatment is carried out after the crystalline metallosilicate is subjected to the series of treatment (A). Carrying out the treatment (B) can improve the thermal stability of the catalyst itself and can lengthen the catalyst life. In particular, carrying out the treatment (B) and setting the supporting amount of the transition metal (X) so as to be specific particularly increases the catalyst activity resulting in significant improvement of the catalyst life.

The metal (Y) is, for example, one kind or two or more kinds of metals selected from the group consisting of alkali metals (Li, Na, K, Rb and Cs), alkaline earth metals (Mg, Ca, Sr and Ba) and rare earth metals (Sc, Y, La, Ce, Pr, Nd, Pm, Sm, Eu, Gd, Tb, Dy, Ho, Er, Tm, Yb and Lu); preferable are alkali earth metals, and more preferable is barium.

As a method for supporting the metal (Y), commonly known methods are used without particular limitation, for example, ion exchange method using a metal salt, evaporative impregnation method, incipient wetness method, pore filling method and solid state supporting method. A preferred method is ion exchange method using a metal salt. The ion exchange method can be carried out for plural times, and the number of carrying out the method is not particularly limited.

The supporting methods may optionally involve the use of a solvent. The solvent, which is usually water or alcohols, is not particularly limited as long as dissolving a metal salt used for the supporting.

### <Treatment of Supporting Transition Metal (X)>

The treatment of supporting the transition metal (X) is a treatment to supporting the transition metal (X) on the modified crystalline metallosilicate. Through this treatment, the metallosilicate catalyst (1) or (2) can be obtained.

The transition metal (X) is not particularly limited, but preferred examples thereof are molybdenum, tungsten and rhenium, and a more preferred example is molybdenum. The use of these metals as the transition metal (X) is preferable because of allowing the activation of the lower hydrocarbon, a raw material, to efficiently proceed. These transition metals (X) may be contained in the transition-metal-containing crystalline metallosilicate catalyst, in a single kind or in two or more different kinds.

As a source of the transition metal (X), every available transition metal compound such as oxides, carbides, acids and salts is employable. In the case of molybdenum, there can be mentioned molybdenum oxide, molybdenum carbide, molybdic acid, sodium molybdate, ammonium molybdate, ammonium heptamolybdate, ammonium paramolybdate, 12-molybdophosphoric acid and 12-molybdosilicic acid.

As a method for supporting the transition metal (X), commonly known methods are used without particular limitation. Examples thereof include a method of supporting a simple substance of the transition metal (X) or a compound containing the transition metal (X) on the modified crystalline metallosilicate, and a method of physically mixing a simple substance of the transition metal (X) or a compound containing the transition metal (X) with the modified crystalline metallosilicate. Preferable is the method of supporting the compound containing the transition metal (X) on the modified crystalline metallosilicate. Specific examples thereof include impregnation method such as pore filling method, incipient wetness method, equilibrium adsorption method, evaporation to dryness method and spray drying method, liquid-phase deposition method, ion exchange method and vapor deposition method. A preferred exemplary method is the impregnation method, which involves relatively simple operation and does not require special equipment.

After the supporting or the mixing step, the transition-metal-containing crystalline metallosilicate catalyst may be calcined in air or in an inert gas such as a nitrogen gas, and may be calcined preferably in air at 250 to 800° C., more preferably at 350 to 600° C., particularly preferably at 450 to 550° C.

In the metallosilicate catalyst (1), the amount of the supported or mixed transition metal (X) with respect to the modified crystalline metallosilicate is 5 to 25 parts by weight, preferably 7 to 25 parts by weight, more preferably 8 to 18 parts by weight based on 100 parts by weight of the modified crystalline metallosilicate. The use of the metallosilicate catalyst (1) in the above production process with the amount of the supported or mixed transition metal (X) in the above range, even though the crystalline metallosilicate is subjected to the series of treatment (A), enables the activation of the lower hydrocarbon, a raw material, and the subsequent aromatization reaction of the activated lower hydrocarbon to proceed with good balance, and moreover can lengthen the catalyst life thanks to improvement of the thermal stability of the catalyst itself.

In the metallosilicate catalyst (2), the amount of the supported or mixed transition metal (X) with respect to the modified crystalline metallosilicate is usually 0.1 to 50 parts by weight, preferably 0.2 to 30 parts by weight, more preferably 1 to 20 parts by weight, particularly preferably 5 to 20 parts by weight based on 100 parts by weight of the modified crystalline metallosilicate. The amount of the supported or mixed transition metal (X) within the above range enables the activation of the lower hydrocarbon, a raw material, and the subsequent aromatization reaction of the activated lower hydrocarbon to proceed with good balance, efficiently generating the aromatic hydrocarbon.

### [Process for Producing Aromatic Hydrocarbon]

The first and second processes for producing an aromatic hydrocarbon of the present invention comprise the reaction between the transition-metal-containing crystalline metallosilicate catalyst and a lower hydrocarbon containing methane as a major component.

### <Lower Hydrocarbon>

The lower hydrocarbon contains methane usually in an amount of 50% by volume or more, preferably 70% by volume or more, more preferably 80% by volume or more. Exemplary other components than methane contained in the lower hydrocarbon are hydrocarbons having 2 to 6 carbon atoms with specific examples thereof including alkanes such as ethane and propane, and alkenes such as ethylene and propylene.

Methane is contained, for example, in natural gas, in associated gas produced with crude oil, refining cracking off-gas in petroleum refinery industry and petrochemical industry, and in so-called unconventional natural gas such as methane hydrate and biomass gas, etc. These gases are directly used, or are used as a mixture with another gas. Alternatively, these are used after a part thereof is separated and removed thereby controlling the composition.

The lower hydrocarbon preferably does not contain a substance which may deteriorate the activity of the catalyst. Prior to the step of supplying the lower hydrocarbon into the reactor, a step may be incorporated of separating and removing compounds containing nitrogen, sulfur and phosphorus, a large amount of water, hydrogen, carbon monoxide and carbon dioxide thereby controlling the concentration. However, the lower hydrocarbon may contain components such as nitrogen, helium, argon, oxygen, carbon monoxide and hydrogen in a range which does not affect the effect of the present invention.

Exemplary aromatic hydrocarbons produced from the lower hydrocarbon are monocyclic aromatic hydrocarbons such as benzene, toluene and xylene and polycyclic aromatic hydrocarbons such as naphthalene and methyl naphthalene.

### <Reaction Conditions and Reaction Equipment>

The reaction temperature (catalyst bed temperature) is usually 600 to 950° C., preferably 650 to 800° C., more preferably 700 to 750° C. The reaction pressure may be any of normal pressure, increased pressure and reduced pressure, but is usually about 0.1 to 0.8 megapascal (MPa), preferably about 0.1 to 0.4 MPa, more preferably about 0.1 to 0.3 MPa, particularly preferably about 0.1 to 0.2 MPa.

The reaction may be carried out by adding an inert gas along with the lower hydrocarbon, a reaction raw material, thereby diluting the inside of the system. Exemplary inert gases are nitrogen, helium and argon.

Exemplary reaction equipment modes are any mode such as a fixed bed, a fluidized bed, a moving bed, a transporting bed, a circulating fluidized bed and combinations thereof.

In the present invention, the reaction may be preceded by a treatment of activating the catalyst. Specifically, there can be mentioned a process in which at least one gas selected from a lower hydrocarbon and a hydrogen gas is contacted preliminarily with the catalyst at a temperature lower than a reaction temperature, and then the catalyst is contacted with the lower hydrocarbon containing methane as a major component.

The transition-metal-containing crystalline metallosilicate catalyst has a high durability under high temperature conditions, i.e., thermal stability and a long catalyst life. Thus, the use of the transition-metal-containing crystalline metallosilicate catalyst can efficiently produce the aromatic hydrocarbon.

### [Transition-Metal-Containing Crystalline Metallosilicate Catalyst]

As described in Background Art, it was difficult even for a skilled person in the art to apply the modified crystalline metallosilicate subjected to the series of treatment (A) to the reaction to produce the aromatic hydrocarbon from methane and achieve a further improvement. The present inventors diligently studied the above use and improvement difficult to conceive for the skilled person, and found that the catalyst obtainable by subjecting the crystalline metallosilicate to the series of treatment (A) and further the treatment (B), and the catalyst obtainable by supporting a specific amount of the transition metal (X) on the modified crystalline metallosilicate are suitable for the above reaction and exhibit their performances. The present invention has been made based on this finding.

Specifically, the transition-metal-containing crystalline metallosilicate catalyst of the present invention is the metallosilicate catalyst (1) or (2) as described above, and this catalyst is used as a catalyst for the process for producing the aromatic hydrocarbon of the present invention as described above, specifically, for the catalytic reaction of the lower hydrocarbon containing methane as a major component.

### EXAMPLES

Hereinafter, the present invention is described in more detail with reference to Examples, but the present invention is not limited by these Examples.

### Catalyst Preparation Example 1

4.0 g of an ammonium type ZSM-5 zeolite (manufactured by Zeolyst) having a silica/alumina ratio of 30 was calcined in air at 500° C. for 4 hours, thereby obtaining a proton type zeolite [a].

### Catalyst Preparation Example 2

### Treatment of Contacting with Aqueous Solution of Ammonium Hexafluorosilicate

11 g of an ammonium type ZSM-5 zeolite (manufactured by Zeolyst) having a silica/alumina ratio of 30 was soaked in 50 mL of distilled water and deaerated at room temperature under reduced pressure. Into the mixture liquid having this zeolite soaked therein, a solution obtained by dissolving 6.8 g of ammonium hexafluorosilicate in 300 mL of distilled water was gradually added at room temperature, and the mixture thus obtained was stirred at 90° C. for 17 hours. The mixture was cooled to room temperature, and subjected to filtration, washing with distilled water and drying successively. Then, the resultant was calcined in air at 500° C. for 4 hours, thereby obtaining a modified zeolite [A].

Elemental analysis of this modified zeolite [A] was carried out by Inductively Coupled Plasma Atomic Emission Spectroscopy (ICP-AES), and found that the silica/alumina ratio was 56.

Elemental analysis of a filtrate obtained by the filtration, including washings, after the treatment of contacting with the aqueous solution of ammonium hexafluorosilicate, found the inclusion of 3.7 mmol of aluminum and 2.7 mmol of silicon. In view of the amount of silicon contained in the added ammonium hexafluorosilicate being 38 mmol, it was made clear that the treatment of contacting with the aqueous solution of ammonium hexafluorosilicate caused the elimination of 3.7 mmol of aluminum from 11 g of the ammonium type ZSM-5 zeolite, and the fixing of 35 mmol of silicon through silylation. It was thus confirmed that the treatment of contacting with the aqueous solution of ammonium hexafluorosilicate caused the elimination of part of aluminum and silylation by one-pot.

### Catalyst Preparation Example 3

### Treatment of Supporting Barium Through Ion Exchange Method

4.0 of the modified zeolite [A] obtained in Catalyst Preparation Example 2 was soaked in 50 mL of distilled water and deaerated at room temperature under reduced pressure. Into the mixture liquid, a solution obtained by dissolving 49.7 g of barium chloride dihydrate in 150 mL of distilled water was gradually added at room temperature, and the mixture thus obtained was stirred at 80° C. for 2 hours, thereby supporting barium by ion exchange method. The mixture was cooled to room temperature, and subjected to filtration, washing with distilled water and drying successively. Then, the resultant was calcined in air at 500° C. for 4 hours, thereby obtaining a barium-supported modified zeolite [B] which supported a barium ion after the treatment with ammonium hexafluorosilicate.

### Catalyst Preparation Example 4

The same procedure was carried out as in Catalyst Preparation Example 3, except for using an ammonium type ZSM-5 zeolite (manufactured by Zeolyst) having a silica/alumina ratio of 30 instead of the modified zeolite [A], thereby obtaining a barium-supported zeolite [b] which had been subjected only to the treatment of supporting barium by ion exchange method.

### Catalyst Preparation Example 5

### Heat Treatment

In order to compare and analyze the durability against high temperature conditions, the zeolite [a] was subjected to a heat treatment as described below.

2.0 g of the zeolite [a] was packed into a quarts tube, and allowed to stand at 750° C. for 3 days under helium flowing. Then, after the temperature was decreased to room temperature, the catalyst was collected, thereby obtaining a zeolite [aH].

In addition to the zeolite [a], the same treatment was carried out also with respect to the modified zeolite [A], the barium-supported modified zeolite [B] and the barium-supported zeolite [b], thereby obtaining a modified zeolite [AH], a barium-supported modified zeolite [BH] and a barium-supported zeolite [bH].

### Catalyst Preparation Example 6

### Supporting of Molybdenum

Ammonium heptamolybdate ((NH₄)₆Mo₇O₂₄.4H₂O, manufactured by Wako Pure Chemical Industries, Ltd.) was dissolved in ion exchange water. Ammonium heptamolybdate was used in such an amount that molybdenum would be supported by a catalyst to be prepared in an amount of 12% by weight (in this case, 14 parts by weights based on 100 parts by weight of the zeolite [a]). Into the solution, 5.0 g of the zeolite [a] was suspended, and the suspension was stirred for a while, and then dried at 120° C. and calcined at 500° C., thereby obtaining a molybdenum-supported catalyst [Mo-a].

In addition to the zeolite [a], the same treatment was carried out also with respect to the zeolite [aH], the modified zeolites [A] and [AH], the barium-supported modified zeolites [B] and [BH], and the barium-supported zeolites [b] and [bH], thereby obtaining molybdenum-supported catalysts [Mo-aH], [Mo-A], [Mo-AH], [Mo-B], [Mo-BH], [Mo-b] and [Mo-bH]. The amount of supported molybdenum is the same as in the case of the zeolite [a].

### Example 1

Using methane as a reaction gas, a catalytic performance was evaluated as described below with a fixed-bed flow reactor.

0.3 g of the catalyst [Mo-A] was packed into a reaction tube, and then the temperature was raised to 200° C. under helium flowing. Then, a mixed gas of methane and hydrogen (a molar ratio of methane to hydrogen was 1:10) was flown, and the temperature was raised to 700° C. The temperature was kept at 700° C. for 80 minutes, and then the flowing was switched to methane, which was a reaction gas, (7.5 mL/min), thereby starting the reaction at 700° C. at normal pressure. A gas at the exit of the reactor was introduced online to a gas chromatograph (GC14A manufactured by Shimadzu Corporation) for analysis.

A yield of benzene was calculated from the following formula (1).

In addition, a preservation ratio of the benzene yield was calculated from the following formula (2).

Yield of benzene (%)=100×Amount of benzene generated (mol)×6/Amount of methane fed (mol) (1)

Preservation ratio of the benzene yield (%)=100×(Yield(18.5 hr))/(Yield(2.5 hr)) (2)

The benzene yield was 6.9% at 2.5 hours from the start of the reaction, and was 5.7% at 18.5 hours from the start of the reaction. The preservation ratio of the benzene yield therebetween was as high as 83%.

Similarly, a catalytic performance of the catalyst [Mo-AH], prepared through high temperature treatment, was evaluated. The benzene yield was as high as 7.3% at 2.5 hours from the start of the reaction, and was 5.9% at 18.5 hours from the start of the reaction. The preservation ratio of the benzene yield was 81%.

A change caused by the heat treatment of the preservation ratio of the benzene yield was calculated from the following formula (3).

Change caused by the heat treatment of the preservation ratio of the benzene yield (%)=100×(Preservation ratio of benzene yield (after heat treatment))/(Preservation ratio of benzene yield (before heat treatment)) (3)

The change caused by the heat treatment of the preservation ratio of the benzene yield was 97%.

The benzene yield and the change caused by the heat treatment of the preservation ratio of the benzene yield are set forth in Table 1.

These results demonstrate that carrying out the treatment with hexafluorosilicates and setting the amount of supported molybdenum so as to be specific improved the durability against high temperature conditions of the transition-metal-containing crystalline metallosilicate catalyst and enhanced the catalyst life.

### Comparative Example 1

A catalytic performance was evaluated in the same manner as in Example 1, except for using the catalysts [Mo-a] and [Mo-aH] instead of the catalysts [Mo-A] and [Mo-AH].

The benzene yield and the change caused by the heat treatment of the preservation ratio of the benzene yield are set forth in Table 1.

In the case of the catalyst [Mo-a] prepared without the heat treatment at high temperature, the benzene yield was 7.0% at 2.5 hours from the start of the reaction, and was 6.5% at 18.5 hours from the start of the reaction. The preservation ratio of the benzene yield was 93%. On the other hand, in the case of the catalyst [Mo-aH] prepared through the heat treatment at high temperature, the benzene yield was 7.3% at 2.5 hours from the start of the reaction, and was 5.3% at 18.5 hours from the start of the reaction. The preservation ratio of the benzene yield was 73%. The change caused by the heat treatment of the preservation ratio of the benzene yield was 78%.

From these results, it is clear that the zeolite [aH], prepared by subjecting the zeolite [a] to high temperature treatment, has inferior performance as a catalyst carrier, and the zeolite [a] has inferior durability against high temperature conditions.

### Example 2

A catalytic performance was evaluated in the same manner as in Example 1, except for using the catalysts [Mo-B] and [Mo-BH] instead of the catalysts [Mo-A] and [Mo-AH].

The benzene yield and the change caused by the heat treatment of the preservation ratio of the benzene yield are set forth in Table 1.

In the case of the catalyst [Mo-B] prepared without the heat treatment at high temperature, the benzene yield was 7.8% at 2.5 hours from the start of the reaction, and was as high as 6.4% at 18.5 hours from the start of the reaction. The preservation ratio of the benzene yield was as high as 82%. On the other hand, in the case of the catalyst [Mo-BH] prepared through the heat treatment at high temperature, the benzene yield was 7.6% at 2.5 hours from the start of the reaction, and was as high as 6.2% at 18.5 hours from the start of the reaction. The preservation ratio of the benzene yield was as high as 82%, as was the case with the catalyst [Mo-B]. The change caused by the heat treatment of the preservation ratio of the benzene yield was 99%.

These results demonstrate that carrying out the treatment with hexafluorosilicates and the subsequent treatment with barium enabled the benzene yield to be kept higher with stability, and further improved the durability against high temperature conditions.

### Comparative Example 2

A catalytic performance was evaluated in the same manner as in Example 1, except for using the catalysts [Mo-b] and [Mo-bH] instead of the catalysts [Mo-A] and [Mo-AH].

The benzene yield and the change caused by the heat treatment of the preservation ratio of the benzene yield are set forth in Table 1.

In the case of the catalyst [Mo-b] prepared without the heat treatment at high temperature, the benzene yield was 7.2% at 2.5 hours from the start of the reaction, and was 5.9% at 18.5 hours from the start of the reaction. The preservation ratio of the benzene yield was 82%. On the other hand, in the case of the catalyst [Mo-bH] prepared through the heat treatment at high temperature, the benzene yield was 6.9% at 2.5 hours from the start of the reaction, and was 5.2% at 18.5 hours from the start of the reaction. The preservation ratio of the benzene yield was 75%, which was lower compared with the catalyst [Mo-b]. The change caused by the heat treatment of the preservation ratio of the benzene yield was 92%.

TABLE 1

| | | | | Change caused by heat |
|---|---|---|---|---|
| | | Benzene yield | | treatment of preservation |
| | Catalyst | 2.5 hours | 18.5 hours | ratio of benzene yield |
| Example 1 | Mo-A | 6.9% | 5.7% | 97% |
| | Mo-AH | 7.3% | 5.9% | |
| Comparative | Mo-a | 7.0% | 6.5% | 78% |
| Example 1 | Mo-aH | 7.3% | 5.3% | |
| Example 2 | Mo-B | 7.8% | 6.4% | 99% |
| | Mo-BH | 7.6% | 6.2% | |
| Comparative | Mo-b | 7.2% | 5.9% | 92% |
| Example 2 | Mo-bH | 6.9% | 5.2% | |

### INDUSTRIAL APPLICABILITY

According to the present invention, a transition-metal-containing crystalline metallosilicate catalyst having a high durability under high temperature condition (namely, thermal stability) and a long catalyst life is obtained economically through a simplified operation, and by using this catalyst, an aromatic hydrocarbon can be produced from a lower hydrocarbon containing methane as a major component while maintaining high yield for long hours, and therefore the above process is industrially suitable.

1. A process for producing an aromatic hydrocarbon comprising the step of reacting a lower hydrocarbon containing methane as a major component in the presence of a transition-metal-containing crystalline metallosilicate catalyst which is obtainable by supporting 5 to 25 parts by weight of a transition metal (X) on 100 parts by weight of a modified crystalline metallosilicate obtainable by subjecting crystalline metallosilicate to a series of treatment (A) comprising a step (i) of eliminating part of a metal from the crystalline metallosilicate and a silylation step (ii).

2. The process for producing an aromatic hydrocarbon according to claim 1, wherein the series of treatment (A) is contacting the crystalline metallosilicate to an aqueous solution of a hexafluorosilicate.

3. The process for producing an aromatic hydrocarbon according to claim 2, wherein the hexafluorosilicate is ammonium hexafluorosilicate.

4. A process for producing an aromatic hydrocarbon comprising the step of reacting a lower hydrocarbon containing methane as a major component in the presence of a transition-metal-containing crystalline metallosilicate catalyst which is obtainable by supporting a transition metal (X) on a modified crystalline metallosilicate obtainable by subjecting a crystalline metallosilicate to a series of treatment (A) comprising a step (i) of eliminating part of a metal from the crystalline metallosilicate and a silylation step (ii) and a treatment (B) of supporting one kind or two or more kinds of metals (Y) selected from the group consisting of alkali metals, alkaline earth metals and rare earth metals.

5. The process for producing an aromatic hydrocarbon according to claim 4, wherein the series of treatment (A) is contacting the crystalline metallosilicate to an aqueous solution of a hexafluorosilicate.

6. The process for producing an aromatic hydrocarbon according to claim 5, wherein the hexafluorosilicate is ammonium hexafluorosilicate.

7. The process for producing an aromatic hydrocarbon according to any one of claims 4 to 6, wherein the treatment (B) is performed through ion exchange method.

8. The process for producing an aromatic hydrocarbon according to any one of claims 4 to 7, wherein the metal (Y) is an alkaline earth metal.

9. The process for producing an aromatic hydrocarbon according to any one of claims 4 to 8, wherein the metal (Y) is barium.

10. The process for producing an aromatic hydrocarbon according to any one of claims 1 to 9, wherein the crystalline metallosilicate is a MFI-type zeolite or a MWW-type zeolite.

11. The process for producing an aromatic hydrocarbon according to any one of claims 1 to 10, wherein the transition metal (X) is one kind or two or more kinds selected from the group consisting of molybdenum, tungsten and rhenium.

12. The process for producing an aromatic hydrocarbon according to any one of claims 1 to 11, wherein the transition metal (X) is molybdenum.

13. A transition-metal-containing crystalline metallosilicate catalyst for use in the process for producing an aromatic hydrocarbon as described in claim 1, which catalyst is obtainable by supporting 5 to 25 parts by weight of a transition metal (X) on 100 parts by weight of a modified crystalline metallosilicate obtainable by subjecting a crystalline metallosilicate to a series of treatment (A) comprising a step (i) of eliminating part of a metal from the crystalline metallosilicate and a silylation step (ii).

14. A transition-metal-containing crystalline metallosilicate catalyst for use in the process for producing an aromatic hydrocarbon as described in claim 4, which catalyst is obtainable by supporting a transition metal (X) on a modified crystalline metallosilicate obtainable by subjecting a crystalline metallosilicate to a series of treatment (A) comprising a step (i) of eliminating part of a metal from the crystalline metallosilicate and a silylation step (ii) and a treatment (B) of supporting one kind or two or more kinds of metals (Y) selected from the group consisting of alkali metals, alkaline earth metals and rare earth metals.
